(19) United States
(12) Patent Application Publication
(10) Pub. No.: US 2004/0247576 A1
(43) Pub. Date: Dec. 9, 2004

(54) Systems and methods for treating vein grafts

(75) Inventor: Axel F. Brisken, Fremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PHARMASONICS, INC., Sunnyvale, CA

(21) Appl. No.: 10/454,828

(22) Filed: Jun. 3, 2003

(51) Int. Cl. A61K 48/00; C12N 15/85

(52) U.S. Cl. 424/93.21; 435/173.1; 435/455

(57) ABSTRACT

Methods, systems and devices are provided which promote the uptake of one or more agents into cellular material, wherein incorporation of such agent(s) improve the intended performance of the material upon implantation. This is particularly applicable to vascular replacements wherein uptake of agents, such as nucleic acids, induces long-term stable adaptation of the vascular replacement involving resistance to neointimal hyperplasia and atherosclerosis. The promotion of such uptake is achieved with the use of vibrational energy, particularly ultrasound. The cellular material is immersed into an acoustically transmissive solution comprising the agent. The immersed material is then exposed to vibrational energy under conditions and for a time which promotes incorporation of the agent into the cells. Portable, convenient and easy to use systems and devices are also provided for use in conjunction with these methods.

### CROSS-REFERENCES TO RELATED APPLICATIONS

Not Applicable

### STATEMENT AS TO RIGHTS TO INVENTIONS MADE UNDER FEDERALLY SPONSORED RESEARCH OR DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK.

Not Applicable

### BACKGROUND OF THE INVENTION

The present invention relates generally to medical devices and methods. More particularly, the present invention relates to methods and apparatus for inhibiting neointimal hyperplasia in vascular replacements used to treat atherosclerotic disease.

The most common type of atherosclerotic disease is coronary atherosclerosis which causes significant morbidity and mortality in the industrialized western world. Coronary atherosclerosis occurs when plaque accumulates on the walls of the coronary arteries narrowing the space for blood to flow through. This can lead to an increased risk of blood clots and an enlarged heart due to the extra pumping effort required. The management of patients suffering from coronary atherosclerosis was dramatically changed by the development of coronary artery bypass grafting (CABG) which was developed in the 1960s and 1970s. CABG reroutes blood around narrowed or blocked arteries to areas of the heart that are not receiving enough blood. This is achieved by attaching a conduit to the aorta above the blockage and to the coronary artery below the blockage. Blood flows through the conduit, going around the blockage in the coronary artery—thus the term “bypass.” This improves the supply of blood and oxygen to the heart to reduce the incidence of ischemia.

A common material used to build this new pathway is a vein from the lower extremity. A long straight vein called the greater saphenous vein runs from just inside the ankle bone up to the groin. This vein is just one of a large series of veins in the lower extremity. However, the greater saphenous vein is the right size, shape, and length for use as a bypass conduit. Although routinely applied and ubiquitously used, vein grafting is not without significant constraints and complications. Venous conduits lack vasomotor tone and are prone to thrombotic and hyperplastic occlusion and, less frequently, infection. Other types of conduits have also been used, such as native arterial vessels, synthetic grafts, endothelial cell lined synthetic grafts and tissue engineered vessels composed of biological materials and autologous cells. While many of these procedures have gained wide acceptance, they continue to suffer from the subsequent occurrence of stenosis or occlusion, often due to intimal hyperplasia.

Intimal hyperplasia is tissue ingrowth into the luminal space of the vessel wall, often due to graft-induced mechanical injury and inflammatory response. Intimal hyperplasia is a direct result of injury-activated smooth muscle cell (SMC) interactions with the vessel wall extracellular matrix and graft materials. In many cases it has been found that the anastomotic junction, which is created at each end of the graft where the graft joins with the native artery, is at significant risk of occlusion due to hyperplasia. The intimal hyperplasia of the vascular SMCs often occurs as an injury response to the surgical creation of the anastomosis. Occlusion resulting from the hyperplasia is exacerbated by thrombosis which occurs as a result of the blood flow turbulence at the site of the anastomosis.

When an anastomotic junction from a CABG procedure fails, it is possible for the heart bypass patient to have the procedure redone, however second and later procedures are seldom as effective in treating the disease as in an initial bypass procedure. Moreover, the availability of autologous blood vessels for performing the procedure places a limit on the number of procedures that can be performed using native vessels.

Vascular grafts are also used in other surgical procedures where intimal hyperplasia may lead to complications. For example, a vascular graft is used to create arterio-venous (A-V) fistula for hemodialysis. The A-V fistula is classically defined as an abnormal vascular connection allowing the flow of blood from an artery directly to a vein. While this type of fistula does not naturally occur in the human body, such a connection is created by a vascular graft for use in hemodialysis. Chronic kidney failure patients must have their blood filtered through a machine (hemodialysis) every two or three days which requires a viable artery and vein to draw blood and then to subsequently return the de-toxified blood. The fistula may be created from a native vessel or an artificial graft. When the anastomotic junction in the A-V fistula fails in the dialysis patient, it is necessary create a new dialysis access site. After a time, there are no more new sites and kidney dialysis is no longer available to the patient.

Numerous pharmacologic approaches have attempted to inhibit neointimal hyperplasia in blood vessels with varied success. However, a growing understanding of the molecular biology of vascular cell activation and proliferation has allowed the design of interventions at the level of gene expression that have successfully altered the course of disease in vein replacements. Blockade of cell-cycle regulatory genes through intraoperative transfection of a combination of anti-sense oligodeoxynucleotides have been shown to inhibit neointimal hyperplasia in rabbit vein grafts for up to 10 weeks after implantation. Although such results are promising, further treatment improvements might arise from transfection rate enhancement.

Thus, it would be desired to provide an improved treatment for intimal hyperplasia resulting from grafting procedures. Particularly, methods and apparatus that would at least partially inhibit excessive cell proliferation of vascular smooth muscle cells in the neointimal layer which forms following a primary treatment and which can result in hyperplasia and subsequent restenosis of the blood vessel. Further, it would be desired to provide methods and apparatus that would increase the transfection rate of nucleic acids, such as DNA, RNA, oligonucleotides, proteins, peptides, small molecule drugs and other species. It would be particularly desirable to provide methods and apparatus that would improve the long-term outcome of the procedure yet minimize the additional tools or steps performed in the surgical procedure to achieve these improved results. Thus, the apparatus would be portable, convenient and easy to use with minimal time, cost and complexity involved in its use. At least some of these objectives will be met by the invention described hereinafter.

### BRIEF SUMMARY OF THE INVENTION

The present invention provides methods, systems and devices for treating cellular material prior to implantation into a patient in an effort to improve the intended performance of the implanted material. In particular, the present invention promotes the uptake of one or more agents into the cellular material, wherein incorporation of such agent(s) improve the intended performance of the implanted material. The present invention is particularly applicable to vascular replacements wherein uptake of agents, such as nucleic acids, induces long-term stable adaptation of the vascular replacement involving resistance to neointimal hyperplasia and atherosclerosis. The promotion of such uptake is achieved with the use of vibrational energy, particularly ultrasound. The cellular material is immersed into an acoustically transmissive solution comprising the agent. The immersed material is then exposed to vibrational energy under conditions and for a time which promotes incorporation of the agent into the cells. Portable, convenient and easy to use systems and devices are also provided for use in conjunction with these methods.

The present invention is applicable to any material which includes cells. Thus, the material may comprise a naturally occurring tissue, such as a natural artery, natural vein or natural organ, to name a few. Alternatively, the material may comprise a synthetic material, such as a synthetic vascular graft or synthetic sheet of grafting material, which includes cells or cellular material. Thus, in some embodiments, the cellular material comprises a cell-seeded vascular graft. Further, the material may include a tissue which is human engineered or tissue engineered, wherein biological tissue is created through the use of cells, with the aid of supporting structures and/or biomolecules. Thus, in some embodiments, the cellular material comprises a tissue engineered vascular graft or a tissue engineered organ, to name a few. Likewise, the cellular material may comprise any combination of these.

The cells of the cellular material may be of any source. Thus, the cells may be autologous, heterogeneous, xenogenous, or a combination of these. Further the cells may be transgenic.

Any suitable agent may be used in conjunction with the present invention. The agent may be selected from the group consisting of nucleic acids, proteins and small molecule drugs. The phrase “nucleic acids” includes both DNA and RNA constructs of the type which encode an immunogen, or a fragment or a peptide thereof. Further, nucleic acids include oligonucleotides, particularly E2F decoy oligonucleotides as described in Ehsan, et al. “Long-term Stabilization of Vein Graft Wall Architecture and Prolonged Resistance to Experimental Atherosclerosis after E2F Decoy Oligonucleotide Gene Therapy” in The Journal of Thoracic and Cardiovascular Surgery, Volume, 121, Number 4, pp. 714-722, incorporated herein by reference for all purposes. The term “transfection” refers to the uptake of an external molecule by the cells and the functional combining the external molecule with the DNA of the cell.

The phrase “vibrational energy” refers to ultrasonic, acoustic, and other mechanical forms of vibration which may be applied according to the methods described herein. Usually, the vibrational energy will be ultrasonic energy delivered under the conditions set forth below.

In a first aspect of the present invention, methods are provided for treating the cellular material prior to implantation. In preferred embodiments, the methods include providing cellular material intended for transplantation into a host and immersing the cellular material into an acoustically transmissive solution which includes an agent that can be incorporated into cells of the cellular material. The methods further include exposing the immersed cellular material to vibrational energy under conditions and for a time which promotes incorporation of the agent into the cells. These conditions will be described in detail in later sections.

When the cellular material comprises excised tissue, the embodiments of the methods include providing the excised tissue intended for transplantation into a host, immersing the tissue in an acoustically transmissive solution, said solution comprising an agent which is capable of transfection, and exposing the immersed tissue to vibrational energy under conditions and for a time which promotes uptake of the agent into the tissue. Further, some embodiments further include the steps of excising the tissue and/or transplanting the tissue into the host.

In each of these embodiments, the methods may further comprise mounting the tissue or cellular material on a support so that at least a portion of the material is disposed within a focal zone of the vibrational energy. The focal zone is a region in which the ultrasonic strength is uniform to within approximately 1, 3, or 6 dB. This promotes uniformity of treatment and consistent results. Mounting may comprise positioning the cellular material in a manner so that the material is axially exposed to the vibrational energy. In this position, typically the entire length of the material may be treated at one time. Alternatively, mounting may comprise positioning the cellular material in a manner so that the material is laterally exposed to the vibrational energy. In this position, the focal zone may be moved axially along the length of the cellular material. In this way, the entire length of the material may be treated in the course of a sweep.

In a second aspect of the present invention, systems are provided for treating cellular material prior to implantation. In preferred embodiments, systems include a container for holding a treatment solution, a support for suspending the cellular material within the treatment solution in the container, and a vibrating surface coupled to deliver vibrational energy to the treatment solution within the container. The treatment solution includes one or more agents for incorporation into the cells. Typically, the container includes an inner sleeve, wherein the tissue is suspended in the sleeve which can be filled with the treatment solution. The container also usually includes a rigid outer shell, wherein the inner sleeve is suspendable in the rigid outer shell which can be filled with an acoustically transmissive solution. The acoustically transmissive solution may comprise water, saline or any other bulk solution which facilitates transmission of vibrational energy. Although both the treatment solution and the acoustically transmissive solution transmit vibrational energy, the solutions are separated by the inner sleeve to prevent dilution of the treatment agents to prevent dilution of the treatment agents and to prevent loss of sterility.

In some embodiments, the inner sleeve is axially elongated. This is particularly suitable for the immersion of elongated tissues or cellular materials such as vascular replacements. The vibrating surfaces may be disposed at various positions in relation to the inner sleeve and therefore the immersed tissue within. For example, the vibrating surface may be positioned to direct the vibrational energy in an axial direction substantially parallel to the axis of the inner sleeve. This is typically achieved by positioning the vibrating surface at the base of the outer shell so that the vibrational energy is directed upwards along the longitudinal axis of the inner sleeve. Since the focal zone of the vibrational energy is located at an axial distance from the vibrating surface, it is preferred that the cellular material be positioned within this axial distance from the vibrating surface so that a majority of the material is disposed within the focal zone.

Alternatively, the vibrating surface may be positioned to direct the vibrational energy in a lateral direction substantially perpendicular to the axis of the cellular material. This is typically achieved by positioning the vibrating surface along the wall of the outer shell so that the vibrational energy is directed laterally toward the inner sleeve. In this case, the focal zone of the vibrational energy is located at a lateral distance from the vibrating surface. Thus, it is preferred that the cellular material be positioned within this lateral distance from the vibrating surface so that at least a portion of the material is disposed within the focal zone. In some embodiments, the vibrating surfaces are an axial transducer array disposed parallel to the axis of the inner sleeve. Generally, the axial transducer array focuses a beam of the vibrational energy toward the cellular material and moves the focused beam along an axial length of the cellular material. The axial transducer array may be a linear array, a phased array, a curved array of either type or a segmented array, to name a few. In the case of a linear array, a sub-array of elements may be selected with appropriate timing of the excitation signals to focus ultrasonic energy onto a small section of the sleeve. For the next burst of ultrasound, one element is added to one end of the sub-array and one element is deleted from the opposite end of the sub-array. In such a manner, the beam walks down the length of the array of elements. In other embodiments, the vibrating surface comprises a transducer assembly which translates along an axial path in the container, the axial path being parallel to the axis of the inner sleeve. Similarly, in other embodiments, the cellular material and usually the inner sleeve translate along an axial path while the vibrating surface remains stationary. In any case, the vibrational energy will be delivered by the vibrating surfaces under conditions which will be described in detail in later sections.

Other objects and advantages of the present invention will become apparent from the detailed description to follow, together with the accompanying drawings.

### BRIEF DESCRIPTION OF THE DRAWINGS

FIG. 1 schematically illustrates an exemplary protocol for performing a method according to the present invention.

FIG. 2 illustrates an embodiment of a treatment system wherein the vibrational energy is axially directed.

FIG. 2A depicts in greater detail an embodiment of the support.

FIG. 2B depicts in greater detail an embodiment of the support wherein the tissue mount includes a perfusion lumen.

FIG. 3 illustrates a theoretical axial beam profile of a vibrational surface.

FIGS. 4A-4C illustrate theoretical lateral beam profiles of the vibrational surface of FIG. 3.

FIG. 5 depicts an experimental axial beam profile of a vibrational surface.

FIG. 6 depicts experimental lateral beam profiles of the vibrational surface.

FIG. 7 illustrates an embodiment of a treatment system wherein the vibrational energy is laterally directed.

FIG. 8 illustrates an embodiment of the treatment system wherein the linear array of transducer elements are axially translated.

FIG. 9 illustrates an embodiment of the treatment system wherein the inner sleeve is axially translated.

FIG. 10 illustrates an embodiment wherein the vibrational surface is disposed on a catheter which is inserted within the cellular material.

### DETAILED DESCRIPTION OF THE INVENTION

Overview of General Method

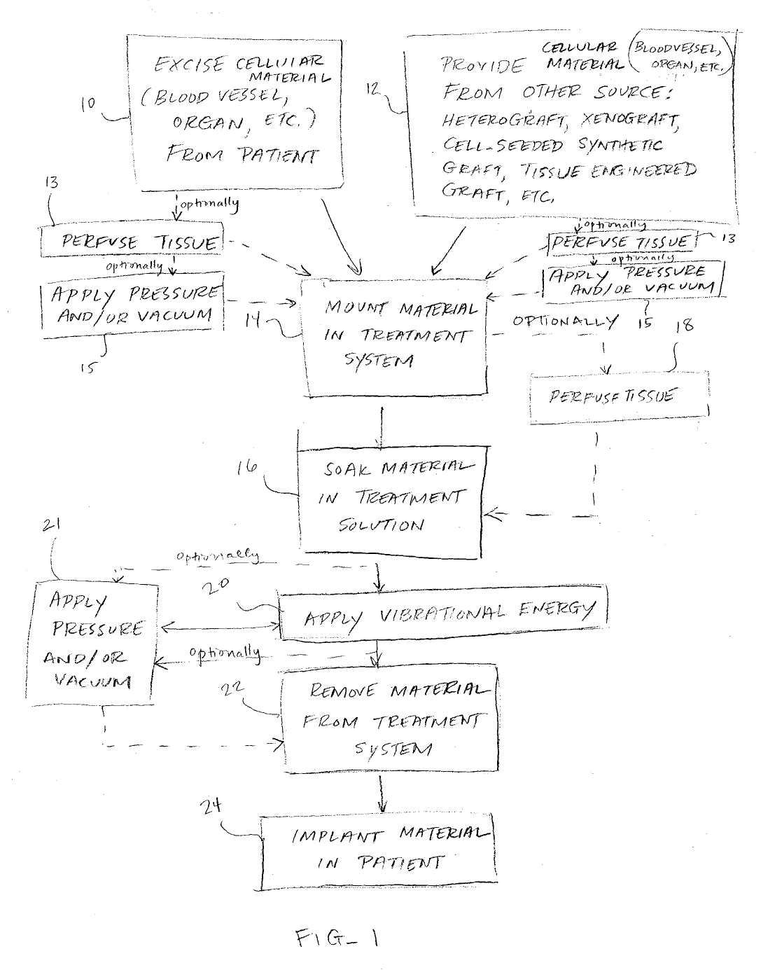

Referring now to FIG. 1, a first exemplary protocol for performing a method according to the present invention is described. To begin, a tissue replacement intended for transplantation into a patient is provided. The tissue replacement may be in any form and will be referred to as cellular material, such as any material which includes cells. Typically the cellular material will be in the form of a tissue. However, the cellular material may also include cells which have not formed a complete tissue structure, such as some cell-seeded grafts. Typically, the cellular material is in the form of a blood vessel, an organ or other tissue body. The cellular material may be provided by the patient or by another source. When the material is provided by the patient, the method includes an excising step 10 wherein tissue is excised from the patient. For example, when the tissue is in the form of a blood vessel, the excising step 10 may involve a procedure to remove a portion of the greater saphenous vein from the leg of the patient. Alternatively, the cellular material may be provided by another source as stated in the providing step 12. When the material is provided by another human, whether living or cadaverous, the material is considered heterogeneous. When the material is provided by another species, the material is considered xenogenous. The cellular material may alternatively be synthetic, typically with the cellular component provided by cell-seeding. For example, the material may be comprised of an endothelial cell seeded vascular graft. Further, the cellular material may be a tissue-engineered material wherein the material is comprised of autologous, heterogeneous, xenogenous, or a combination of these of cells engineered to form a tissue. Other types of material may also be used so long as the material has a cellular component or would somehow benefit from the steps of the method of the present invention.

Optionally, the cellular material may be perfused with solution, perfusion step 13. Perfusion may be achieved by any suitable means so that it passes by or through the tissue. When the cellular material is a tubular vessel, solution may be injected through its inner lumen. Optionally, pressure or vacuum may be applied to the cellular material, step 15. This may assist in the uptake of agents in the solution by the cellular material. The cellular material is then mounted in the treatment system of the present invention, mounting step 14. The treatment system will be described in further detail in later sections. Generally, the treatment system comprises a container holding a treatment solution, wherein the treatment solution contains agents such as nucleic acids, proteins, small molecule drugs or a combination of these, to name a few. The system also includes a support from which the cellular material is suspended so that the material is submerged in the treatment solution. Thus, when the material is mounted on the support, the material soaks in the treatment solution, soaking step 16. Optionally, the cellular material may also be perfused with solution, perfusion step 18. This perfusion solution may be the same or different from prior perfusion solution and/or the soaking treatment solution. Perfusion may be achieved by injecting solution through the support so that it passes by or through the mounted tissue. When the cellular material is a tubular vessel, solution may be injected through its inner lumen. Both the treatment solution and the perfusion solutions are acoustically transmissive.

Vibrational energy is then applied, application step 20, to the immersed cellular material under conditions and for a time which promotes uptake of the agent into the tissue. Example conditions and treatment times will be provided in more detail in later sections. Optionally, pressure or vacuum may be applied to the cellular material, step 21, prior to, during and/or after application of vibrational energy. Or, vibrational energy may be applied several times with intermediate or concomitant pressure or vacuum. The cellular material is then removed from the treatment system, step 22, and implanted directly into the patient, step 24. Typically, the cellular material is a blood vessel and is implanted in the patient as part of a CABG procedure.

Treatment System—Axially Directed Vibrational Energy Embodiment

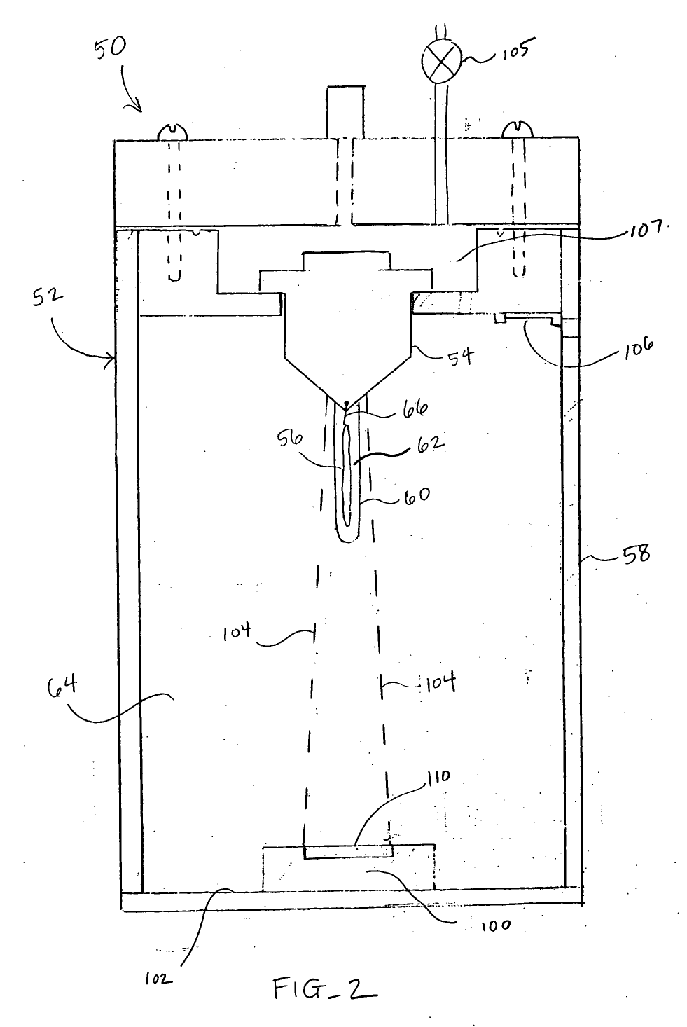

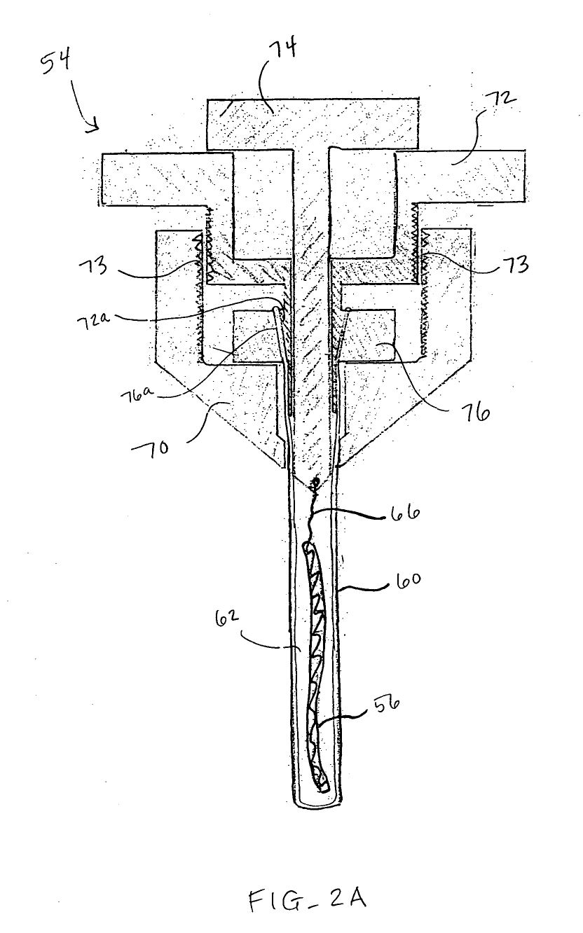

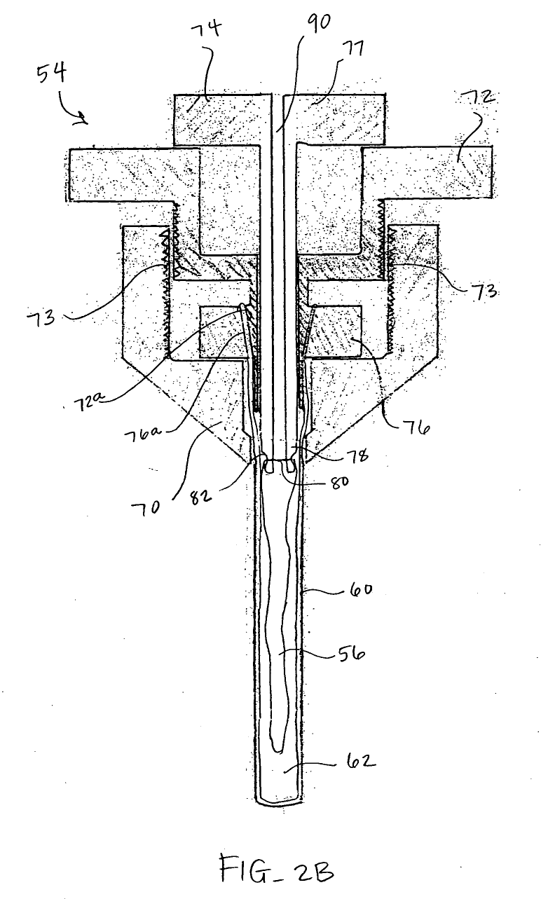

Referring now to FIG. 2, an embodiment of a treatment system 50 of the present invention is illustrated. The treatment system 50 comprises a container 52 for holding solution and a support 54 upon which the cellular material 56 is mounted. In this embodiment, the container 50 includes a rigid outer shell 58 and a flexible inner sleeve 60. The flexible inner sleeve 60 is filled with treatment solution 62 and the sleeve 60 is suspended in the rigid outer shell 58 which is filled with an acoustically transmissive medium 64. As mentioned above, the treatment solution 62 contains agents such as nucleic acids, proteins, small molecule drugs or a combination of these, to name a few. The medium 64 may comprise water or saline or other bulk solution. The treatment solution 62 is held by the flexible inner sleeve 60 to separate the treatment solution 62 from the surrounding acoustically transmissive solution 64. Although both the solutions 62, 64 are acoustically transmissive, the treatment solution 62 is separated from the bulk acoustically transmissive solution 64 to prevent dilution of the treatment agents and to maintain sterility of the cellular material. The sleeve 60 is also acoustically transmissive and is typically fabricated from silicone rubber, latex, or like materials. The sleeve 60 is typically thin walled. The sleeve 60 may also be fabricated from some plastics, such as thin high density polyethylenes, polystyrenes and the like.

The cellular material 56 is mounted on the support 54 to suspend the cellular material 56 in the treatment solution 62, as shown. The cellular material 56 may be mounted on the support 54 by any suitable means, for example, by a suture 66 (shown), hook, fastener, or other means. FIG. 2A depicts in greater detail an embodiment of the support 54. Here, the support 54 includes a clamp base 70, a clamp top 72 and a mount 74. The base 70 and top 72 are joinable by threading 73, as shown, and the mount 74 extends through the syrnnetrical centers of the base 70 and top 72. The flexible inner sleeve 60 is secured between the clamp base 70 and clamp top 72 to hold the sleeve 60 in place and to seal the sterile field of the cellular material 56 and treatment solution 62 from the remainder of the system 54. The sleeve 60 is typically a cylindrical element and is formed or molded from a flexible material, such as latex or silicone rubber, or harder material, such as polystyrene. Typical dimensions for the sleeve 60 are approximately 6 mm in diameter and approximately 4-6 cm in length, however any suitable dimensions may be used, particularly to suit other shaped and/or sized tissue replacements. The sleeve 60 extends between the clamp base 70 and clamp top 72 and is pulled up into the clamp top 72 and held by seal 76. The seal 76 is typically manufactured from plastic but semi-rigid material, such as Dehring, may be used. As the top 72 and base 70 are joined together, the seal 76 is pressed in place and the sleeve 60 is pressed between a slanted surface 76a of the seal 76 and a tapered portion 72a of the top 72. Tightening of the clamp pushes the seal 76 further up the tapered portion 72a to seal the sterile field of the cellular material 56 and treatment solution 62 from the remainder of the system 54.

FIG. 2B similarly depicts in greater detail another embodiment of the support 54. Here, the cellular material 56 is again mounted on the support 54 to suspend the material 56 in the treatment solution 62, as shown. However, in this embodiment, the cellular material 56 is mounted directly on the support 54 so that one end of the material 56 is slipped over a distal end 78 of the mount 74 and secured in place with a tie 80. The distal end 78 may optionally include indentations or grooves 82 over which the tie 80 may be bound to further secure the cellular material 56 in place. In this embodiment, the mount 74 includes a perfusion lumen 90 which extends through the mount 74 from its proximal end 77 to its distal end 78. When the cellular material 56 is a tubular vessel, such as a vein, the material 56 may be secured to the distal end 78 so that the perfusion lumen 90 is aligned with the interior or inner lumen of the tubular vessel. In this manner, perfusion solution may be administered through the perfusion lumen 90 and into the tubular vessel. One reason for providing the perfusion lumen is that it allows the cellular material to be reperfused during the application of vibrational energy, to add new material. This is particularly advantageous if the perfusion solution contains microbubbles, which may be consumed by the ultrasound exposure.

The perfusion lumen 90 may also be used to control the pressure in the inner sleeve 60. A vacuum or a pressurization pump may be applied to the proximal end 77 of the mount 74 to apply a vacuum or pressure, respectively, to the sleeve 60. This may assist in the uptake of the agent by the cellular material 56. Alternatively, as shown in FIG. 2, vacuum or pressurization may be applied to a top chamber 107 within the shell 58 through a valve 105. When the perfusion lumen 90 is open to the top chamber 107, such vacuum or pressure will also be provided to the sleeve 60. It may be appreciated that the top chamber 107 would be sealed to prevent leakage. Thus, as stated previously, pressure or vacuum may be applied to the cellular material prior to, during and/or after application of vibrational energy. Or, vibrational energy may be applied several times with intermediate or concomitant pressure or vacuum.

In FIG. 2, the support 54 both suspends the cellular material 56 in treatment solution 62 contained by sleeve 60, and suspends the sleeve 60 in a bulk acoustically transmissive solution 64 contained by outer shell 58. As mentioned, the acoustically transmissive solution 64 may be comprised of any suitable material, particularly one that facilitates the transmission of ultrasonic energy, provides minimal attenuation of ultrasonic energy and creates minimal nonlinear effects. In preferred embodiments, the solution 64 comprises sterile or nonsterile saline or water. The solution 64 may also include additions such as anti-bacterial agents, anti-fungal agents and/or anti-corrosive agents.

Within the container 50 lies one or more vibrating surfaces 100 coupled to deliver vibrational energy to the treatment solution 62. Typically, the vibrating surfaces 100 lie along the inner surfaces of the outer shell 58 so that the vibrational energy is delivered to the treatment solution 62 through the acoustically transmissive solution 64. In preferred embodiments, the vibrating surfaces 100 might comprise the front surfaces of either quarter wave matched, composite, or Tonpilz transducers. In brief, the quarter wave transducers would typically comprise a planar (or curved) piece of hard piezoelectric, typically PZT-8 or an equivalent, at a thickness of one half the wavelength of the desired operating frequency. The ceramic would be air backed and would further include a quarter wave matching layer on the face. Alternatively, the piezoelectric ceramic might be a PZT-4, or derivatives of this class of materials. The piezo-composite transducer might comprise a 1-3 mode-free structure for large planar apertures or a 2-2 structure for array devices. The Tonpilz transducer might comprise a piezoelectric stack or cylinder between a heavy tail mass and a head mass, where the tail mass would be anchored to the side wall of the outer shell 58 and the head mass would comprise the vibrating surface. Typically the quarter wave and piezo-composite transducers would be preferred for operation at approximately 200 kHz or above while the Tonpilz would be preferred for operation below this frequency.

In FIG. 2, the vibrating surface 100 is mounted along the base 102 of the outer shell 58, directly below the cellular material 56. In this embodiment, the vibrating surface 100 represents the front (or active) surface of the ultrasonic transducer which is excited by an electrical signal from a signal generator and power amplifier. The vibrating front surface generates an ultrasonic beam, the boundary of which is schematically indicated by dashed lines 104, propagating toward the cellular material 56. Ultrasonic energy propagating past or through the sleeve 60 and cellular material 56 is diffused by the support 54. The support 54 reflects the energy in a manner to prevent standing waves. Optionally, an ultrasound sensor 106 may be positioned within the container 50 to monitor the vibrational energy.

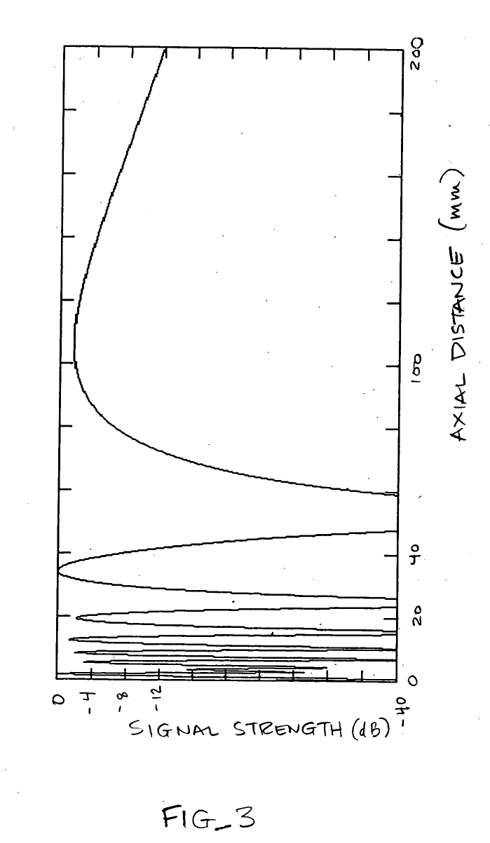

The vibrating surface 100 of the ultrasonic transducer on the base 102 may operate at a frequency of between 30 kHz to 5 MHz, more typically between 300 kHz and 2 MHz, and most typically between 750 kHz and 1.5 MHz. The device might have an aperture 110 of 0.25 to 2.00 inches, more typically 0.75 to 1.25 inches. Further, the device might be spherically curved for focusing, either concave or convex, but more typically flat. A flat ceramic surface 100 with a 1.00 inch diameter operating at 1.0 MHz in a continuous wave mode may have a theoretical axial beam profile as depicted in FIG. 3, wherein the vertical axis depicts signal strength in dB and the horizontal axis reflects the distance from the surface 100 upward toward the cellular material 56. As shown, the ultrasonic strength is uniform to within 1 dB from approximately 85 to 135 mm from the face of the surface 100, a focal zone of approximately 50 mm. Thus, in this embodiment, it is desired that the cellular material 56 be mounted on the support 54 such that the cellular material 56 is located within the focal zone, approximately 85 to 135 mm from the face of the surface 100.

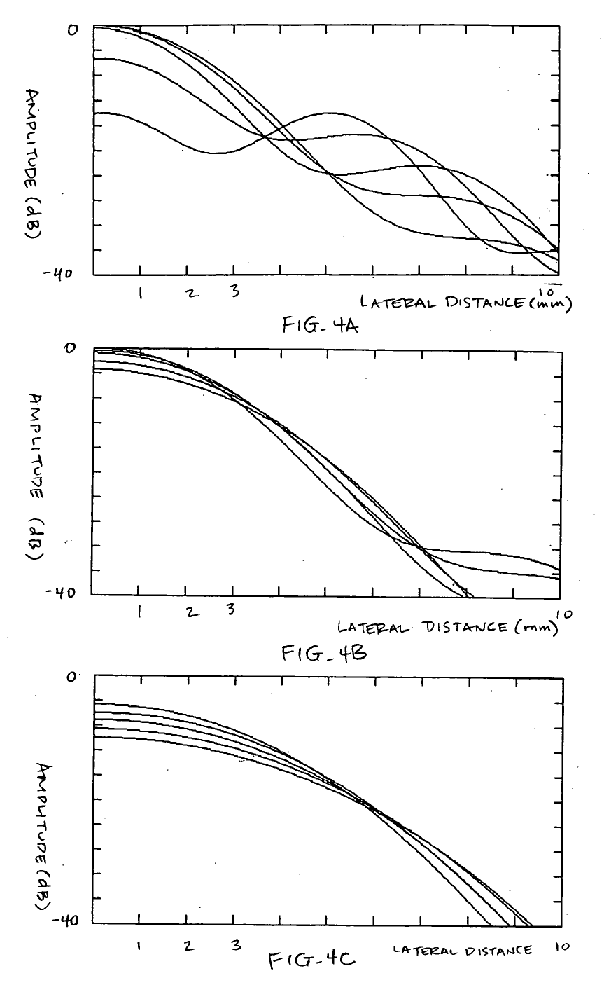

FIGS. 4A-4C depict theoretical lateral beam profiles for the flat ceramic surface 100 with a 1.00 inch diameter operating at 1.0 MHz in a continuous wave mode. FIG. 4A illustrates the lateral beam profiles at axial distances of 70 to 110 mm (in steps of 10 mm) from the surface 100, wherein the broader and weaker profiles correspond to closer distances to the surface 100. FIG. 4B illustrates the lateral beam profiles at axial distances of 120 to 160 mm from the surface 100, where the uniformity of the profiles indicates the uniformity of the beam, and FIG. 4C illustrates the lateral beam profiles at axial distances of 160 to 200 mm, where the beams become weaker and wider with greater distance from the surface 100. Thus, FIGS. 4A-4C represent profiles at locations throughout the focal zone. It can be seen that there exists less than a 3 dB variation in ultrasonic field strength over a width of 6 mm corresponding to axial distances from 90 to 160 mm. Variations in device design can bring about even flatter beam profiles.

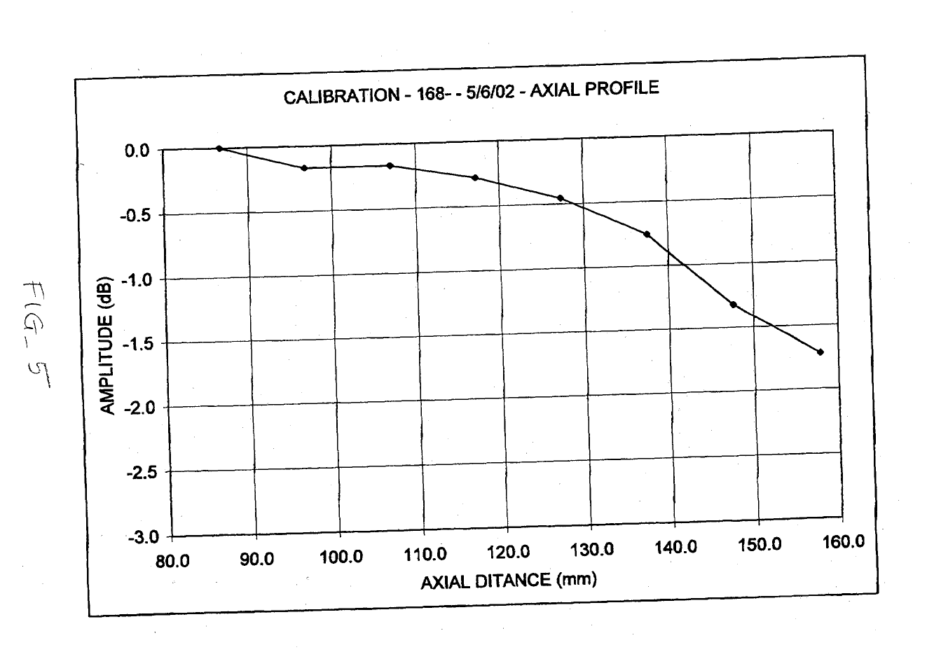

Similarly, FIG. 5 depicts an experimental axial beam profile of a vibrating surface 100 with a 1 inch diameter operating at 1 MHz in a continuous wave mode. Here the vertical axis depicts amplitude in dB and the horizontal axis reflects the axial distance from the surface 100 upward toward the cellular material 56. Again, the ultrasonic strength is uniform to within 1 dB from approximately 85 to 135 mm from the face of the surface 100, a focal zone of approximately 50 mm. Thus, in this embodiment, it is desired that the cellular material 56 be mounted on the support 54 such that the cellular material 56 is located within the focal zone, approximately 85 to 135 mm from the face of the surface 100.

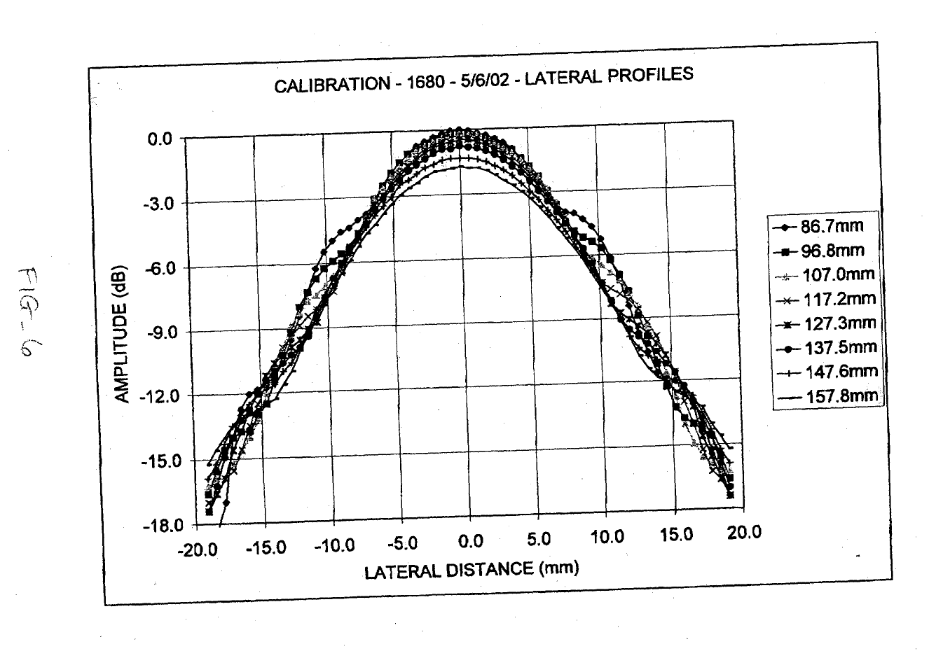

FIG. 6 depicts an experimental lateral beam profiles for the surface 100 with a 1 inch diameter operating at 1 MHz in a continuous wave mode. Each curve represents a lateral beam profile at an axial distance as specified in the legend, ranging from 86.7 mm to 157.8 mm. Thus, FIG. 6 represents profiles at locations throughout the focal zone.

In the above described embodiments, the sleeve 60 will typically attenuate the vibrational energy less than 0.5 dB. Refractive and reflective effects from the sleeve 60 are also minimal. The cellular material 56 itself however might attenuate the vibrational energy. Assuming an attenuation of 0.3 dB per MHz per cm, a 3 cm long portion of cellular material 56 may attenuate the energy a maximum of 1 dB under the most unfortunate conditions. However, this variation in sonication is considered acceptable.

Transducers may be operated in any of the conditions which are effective. These conditions include, but are not limited to the following:

| | | Sample |
|---|---|---|
| Parameter | Range of values | preferred value |
| Frequency | 30 kHz to 5 MHz | 1 MHz |
| Cycles per burst | 2 to 10,000 | 5 |
| Burst rate | 0.1 to 50 kHz | 12 kHz |
| Duty cycle | 0.1 to 50% | 6% |
| Mechanical Index (MI) | 0.5 to 4 | 1.8 |

The frequency is the transmission frequency as defined, for example, by the signal generator of the equipment configuration, an exemplary equipment configuration of which is described and illustrated in copending U.S. patent application No. 09/893341 (Attorney Docket No. 017148-004110US), incorporated herein by reference for all purposes. The number of cycles during the burst, or the cycles per burst, and the burst rate are also defined by the signal generator. The duty cycle is defined as that portion of time during which the power amplifier is energized. The phrase “mechanical index (MI)” is defined as follows: The American Institute for Ultrasound in Medicine (AIUM) and the National Electrical Manufacturers Association (NEMA) in “Standard for Real-Time Display of Thermal and Mechanical Indices on Diagnostic Ultrasound Equipment”, 1991, have together defined the term “mechanical index” MI for medical diagnostic ultrasound systems operating in the frequency range of 1 to 10 MHz. Although therapeutic ultrasound is not included within the scope of this standard, the terms are useful in characterizing ultrasound exposure.

The mechanical index is defined as the peak rarefactional pressure P−(in MPa) at the point of effectivity (corrected for attenuation along the beam path) in the cellular material divided by the square root of the frequency F (in MHz), or

MI=P−[MPa]/sqrt (F [MHz])

The tolerated range for medical diagnostic equipment is up to an MI of 1.9. MI values above approximately unity represent acoustic levels which can cause mechanical bio-effects in human subjects.

The mechanical vibrational energy may be applied for a time sufficient to enhance uptake of the agent, typically from 1 second to 3 minutes, usually from 20 seconds to 80 seconds, although the time of application may vary significantly.

Treatment System—Laterally Directed Vibrational Energy Embodiment

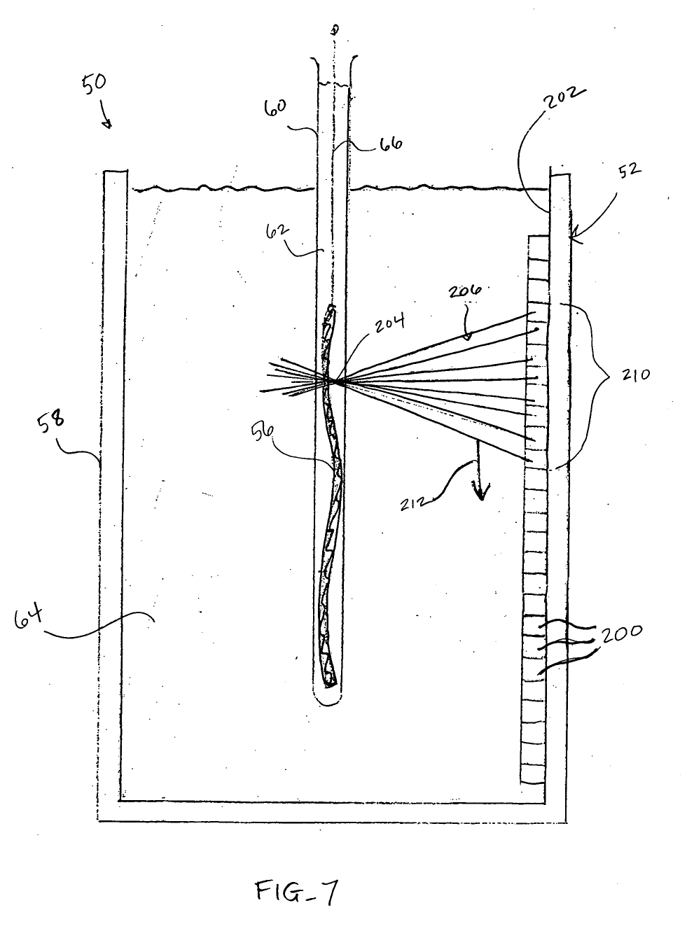

Referring now to FIG. 7, an additional embodiment of a treatment system 50 of the present invention is illustrated. Similar to FIG. 2, the treatment system 50 comprises a container 52 for holding solution and a support 54 upon which the cellular material 56 is mounted. However, for simplicity the support 54 is not illustrated and the flexible inner sleeve 60 is shown extending upward. The sleeve 60 attaches to the support 54 as previously described in relation to FIG. 2. Again, the container 52 includes a rigid outer shell 58 and the inner sleeve 60. The inner sleeve 60 is filled with treatment solution 62 and the sleeve 60 is suspended in the rigid outer shell 58 which is filled with an acoustically transmissive medium 64. As mentioned above, the treatment solution 62 contains agents such as nucleic acids, proteins, small molecule drugs or a combination of these, to name a few. The medium 64 may comprise water or saline or other bulk solution.

In this embodiment, the vibrating surface 100 comprises a linear array of transducer elements 200 positioned along an inner surface of the outer shell 58 so that the cellular material 56 receives vibrational energy in the lateral direction. As shown in FIG. 7, the elements 200 are generally planar and are disposed parallel to the longitudinal axis of the inner sleeve 60, along a side wall 202. In preferred embodiments, subsets of elements 200 are selected sequentially to treat the cellular material 56 inside the sleeve 60. For example, a sub-array 210 of elements 200 are selected, with appropriate timing of the excitation signals, to focus ultrasonic energy onto a small section of the sleeve 60 and therefore the cellular material 56 which lies within. A focal point 204 for the beam 206 is shown positioned along the sleeve 60. For the next burst of ultrasound, one element is added to one end of the sub-array 210 and one element is deleted from the opposite end of the sub-array 210. In such a manner, the beam walks down the length of the array of elements 200, as indicated by arrow 212.

The orthogonal length of the elements 200 may be adjusted to achieve a beam width consistent with the diameter of the cellular material 56 and the beam uniformity desired across the same cellular material. Further, larger diameter discrete transducer elements may alternatively be used which may be switched off and on so as not to interfere with each other.

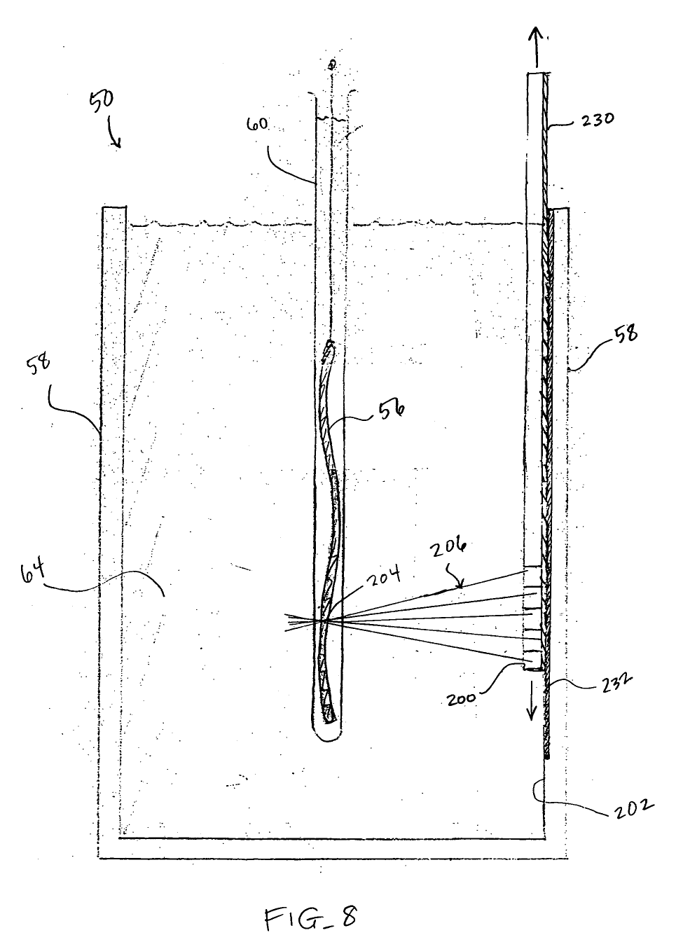

In another embodiment, illustrated in FIG. 8, the linear array of transducer elements 200 focuses the beam onto a small section of the sleeve 60, and therefore the cellular material 56 which lies within, and the beam is moved by movement of the array of elements 200. In this embodiment, five elements 200 are depicted, however, it may be appreciated that any number of elements 200 may be used including a single element transducer with a curved surface to focus in the axial direction and an orthogonal dimension and curvature to cover the cellular material in this same direction. The array of elements 200 are disposed on a transducer assembly 230 which translates along an axial path in the container 50 disposed along the side wall 202 of the outer shell 58. In some embodiments, a track 232 is disposed along the axial path along which the assembly 230 translates. As shown, the focal point 204 for the beam 206 is positioned on the sleeve 60 and the cellular material 56 within. As the array of elements 200 is translated upwards, the focal point 204 will travel up the sleeve 60 and therefore the cellular material 56. Likewise, as the array of elements 200 is translated downwards, the focal point 204 will travel down the sleeve 60 and therefore the cellular material 56. In this manner, vibrational energy may be delivered to the entire length of the cellular material 56.

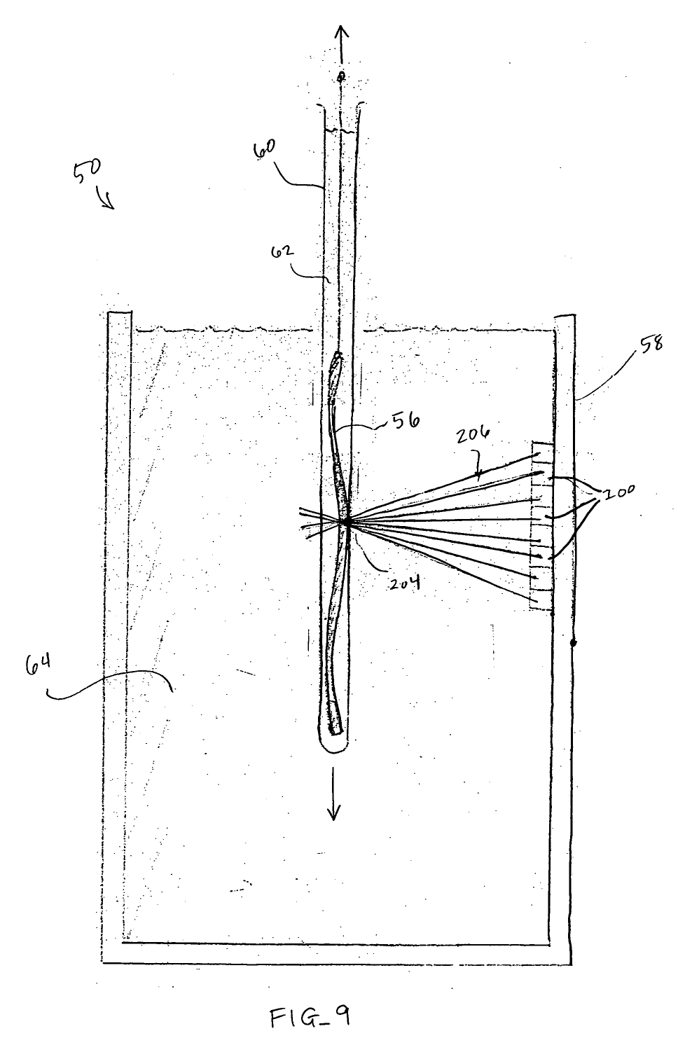

In yet another embodiment, illustrated in FIG. 9, the linear array of transducer elements 200 focuses the beam onto a small section of the sleeve 60, and therefore the cellular material 56 which lies within, and the cellular material 56 is translated axially. In this embodiment, eight elements 200 are depicted, however, it may be appreciated that any number of elements 200 may be used including a single element transducer with a curved surface to focus in the axial direction and an orthogonal dimension and curvature to cover the cellular material in this same direction. The cellular material 56 itself may be translated within the inner sleeve 60 or the cellular material 56 and sleeve 60 together may be translated within the outer shell 58. In either case, the array of elements 200 is disposed along the side wall 202 of the outer shell 58. The focal point 204 for the beam 206 is positioned on the sleeve 60 and the cellular material 56 within. As shown in FIG. 9, as the cellular material 56, and in this embodiment additionally the inner sleeve 60, is translated upwards, the focal point 204 will travel down the sleeve 60 and therefore the cellular material 56. Likewise, as the material 56 and sleeve 60 are translated downwards, the focal point 204 will travel up the sleeve 60 and therefore the cellular material 56. In this manner, vibrational energy may be delivered to the entire length of the cellular material 56.

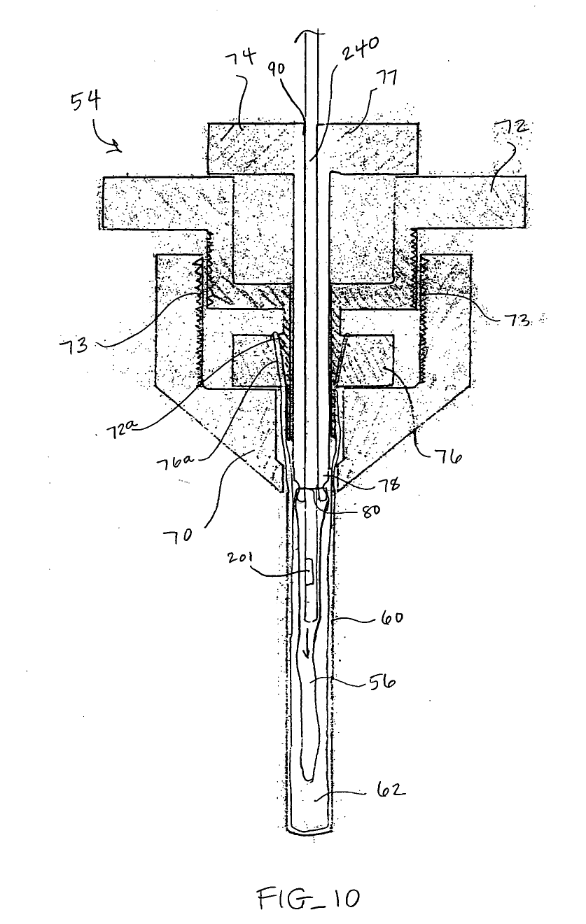

In a further embodiment, illustrated in FIG. 10, a cylindrical transducer element 201 (or multiple elements) disposed on a catheter 240 mm ay be inserted through the central lumen of the cellular material. Such insertion may be achieved by mounting the cellular material 56 on the support 54 as illustrated in FIG. 2B. Here the cellular material 56 is mounted directly on the support 54 so that one end of the cellular material 56 is slipped over a distal end 78 of the mount 74 and secured in place with a tie 80. The catheter 240 may be inserted through the perfusion lumen 90 which extends through the mount 74 from its proximal end 77 to its distal end 78. When the cellular material 56 is a tubular vessel, such as a vein, the cellular material 56 may be secured to the distal end 78 so that the perfusion lumen 90 is aligned with the interior or inner lumen of the tubular vessel. In this manner, catheter 240 may be advanced through the perfusion lumen 90 and into the tubular vessel. As shown, the transducer element 201 is disposed along the catheter substantially parallel to the axis of the inner sleeve 60. Thus, as the catheter 240 is translated upwards, the element 201 travels up the cellular material 56. Likewise, as the catheter 240 is translated downwards, the element 201 travels down the cellular material 56. In this manner, vibrational energy may be delivered radially to the entire length of the cellular material 56.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A method for treating cellular material prior to implantation, said method comprising:
providing cellular material intended for transplantation into a host;
immersing the cellular material into an acoustically transmissive solution, said solution comprising an agent which can be incorporated into cells of the cellular material;
exposing the immersed cellular material to vibrational energy under conditions and for a time which promotes incorporation of the agent into the cells.

2. A method as in claim 1, wherein the cellular material comprises a natural artery, a natural vein, a natural organ, a cell-seeded synthetic vascular graft, a tissue engineered vascular graft, a tissue engineered organ, or a combination of these.

3. A method as in claim 2, wherein the cellular material is autologous.

4. A method as in claim 2, wherein the cellular material is heterogeneous.

5. A method as in claim 1, wherein the agent is selected from the group consisting of nucleic acids, DNA, RNA, oligonucleotides, proteins, peptides and small molecule drugs.

6. A method as in claim 5, wherein the agent includes a nucleic acid and the exposure of vibrational energy promotes transfection.

7. A method as in claim 1, wherein the vibrational energy is applied under conditions comprising a frequency in the range from 30 kHz to 5 MHz and a mechanical index in the range from 0.5 to 4.

8. A method as in claim 7, wherein the conditions further comprise a duty cycle in the range from 0.1% to 50%.

9. A method as in claim 7, wherein the conditions further comprise a burst rate in the range from 0.1 kHz to 50 kHz and cycles per burst in the range from 2 to 10,000.

10. A method as in claim 1, wherein the vibrational energy is applied from a single fixed transducer so that the vibrational intensity varies by less than ±3 dB over the surface of the cellular material.

11. A method as in claim 1, wherein the cellular material includes an inner lumen, said method further comprising perfusing the acoustically transmissive solution comprising the agent through the inner lumen.

12. A method as in claim 1, further comprising mounting the cellular material on a support so that at least a portion of the material is disposed within a focal zone of the vibrational energy.

13. A method as in claim 12, wherein mounting comprises positioning the cellular material in a manner so that the material is axially exposed to the vibrational energy.

14. A method as in claim 12, wherein mounting comprises positioning the cellular material in a manner so that the material is laterally exposed to the vibrational energy.

15. A method as in claim 14, wherein the focal zone is moved axially along the cellular material.

16. A method for treating tissue prior to transplantation, said method comprising:
providing excised tissue intended for transplantation into a host;
immersing the tissue in an acoustically transmissive solution, said solution comprising an agent; and
exposing the immersed tissue to vibrational energy under conditions and for a time which promotes uptake of the agent into the tissue.

17. A method as in claim 16, wherein the excised tissue is a vein intended for transplantation.

18. A method as in claim 17, wherein the vein is autologous.

19. A method as in claim 17, wherein the vein is heterogeneous.

20. A method as in claim 17, wherein the vein is transgenic.

21. A method as in claim 16, wherein the agent is selected from the group consisting of nucleic acids, DNA, RNA, oligonucleotides, proteins, peptides and small molecule drugs.

22. A method as in claim 21, wherein the agent is a nucleic acid and the exposure to vibrational energy promotes uptake and expression of the nucleic acid in the transplanted tissue.

23. A method as in claim 22, wherein the nucleic acid is an antisense oligonucleotide which inhibits gene expression in targets involved in a biological response generating intimal hyperplasia.

24. A method as in claim 16, wherein the vibrational energy is applied under conditions comprising a frequency in the range from 30 kHz to 5 MHz and a mechanical index in the range from 0.5 to 4.

25. A method as in claim 24, wherein the conditions further comprise a duty cycle in the range from 0.1% to 50%.

26. A method as in claim 24, wherein the conditions further comprise a burst rate in the range from 0.1 kHz to 50 kHz and cycles per burst in the range from 2 to 10,000.

27. A method as in claim 16, wherein the vibrational energy is applied from a single fixed transducer so that the vibrational intensity varies by less than ±3 db over the surface of the excised tissue.

28. A method as in claim 16, wherein the vibrational energy is applied from a phased transducer array so that the total vibrational energy delivered to any one location on the excised tissue varies by less than ±3 db.

29. A method as in claim 16, wherein the vibrational energy is applied from a single transducer which translates over a surface of the excised tissue.

30. A method for transplanting tissue to a host, said method comprising:
providing excised tissue intended for transplantation into the host;
immersing the tissue in an acoustically transmissive solution, said solution comprising an agent;
exposing the immersed tissue to vibrational energy under conditions and for a time which promotes uptake of the agent into the tissue; and
transplanting the tissue into the host.

31. A method as in claim 30, wherein the excised tissue is a vein intended for transplantation.

32. A method as in claim 31, wherein the vein is autologous.

33. A method as in claim 31, wherein the vein is heterogeneous.

34. A method as in claim 31, wherein the vein is transgenic.

35. A method as in claim 30, wherein the agent is selected from the group consisting of nucleic acids, proteins, and small molecule drugs.

36. A method as in claim 35, wherein the agent is a nucleic acid and the exposure to vibrational energy promotes uptake and expression of the nucleic acid in the transplanted tissue.

37. A method as in claim 36, wherein the nucleic acid is an antisense oligonucleotide which inhibits gene expression in targets involved in a biological response generating intimal hyperplasia.

38. A method as in claim 30, wherein the vibrational energy is applied under conditions comprising a frequency in the range from 30 kHz to 5 MHz and a mechanical index in the range from 0.5 to 4.

39. A method as in claim 38, wherein the conditions further comprise a duty cycle in the range from 0.1% to 50%.

40. A method as in claim 38, wherein the conditions further comprise a burst rate in the range from 0.1 kHz to 50 kHz and cycles per burst in the range from 2 to 10,000.

41. A method as in claim 30, wherein the vibrational energy is applied from a single fixed transducer so that the vibrational intensity varies by less than ±3 db over the surface of the excised tissue.

42. A method as in claim 30, wherein the vibrational energy is applied from a phased transducer array so that the total vibrational energy delivered to any one location on the excised tissue varies by less than ±3 db.

43. A method as in claim 30, wherein the vibrational energy is applied from a single transducer which translates over a surface of the excised tissue.

44. A system for treating cellular material prior to implantation, said system comprising:
a container for holding a treatment solution;
a support for suspending the cellular material within the treatment solution in the container; and
a vibrating surface coupled to deliver vibrational energy to the treatment solution within the container.

45. A system as in claim 44, wherein the container includes an inner sleeve, wherein the tissue is suspended in the sleeve which can be filled with the treatment solution.

46. A system as in claim 45, wherein the inner sleeve is axially elongated.

47. A system as in claim 46, wherein the vibrating surface is positioned to direct the vibrational energy in an axial direction substantially parallel to the axis of the inner sleeve.

48. A system as in claim 47, wherein the cellular material is positioned an axial distance from the vibrating surface so that a majority of the material is disposed within a focal zone of the vibrational energy.

49. A system as in claim 48, wherein the focal zone is in the range of approximately 85-135 mm.

50. A system as in claim 46, wherein vibrating surface is positioned to direct the vibrational energy in a lateral direction substantially perpendicular to the axis of the cellular material.

51. A system as in claim 50, wherein the cellular material is positioned a lateral distance from the vibrating surface so that at least a portion of the material is disposed within a focal zone of the vibrational energy.

52. A system as in claim 50, wherein the vibrating surface comprises an axial transducer array disposed parallel to the axis of the inner sleeve.

53. A system as in claim 52, wherein the axial transducer array focuses a beam of the vibrational energy toward the cellular material and moves the focused beam along an axial length of the cellular material.

54. A system as in claim 53, wherein the axial transducer array is a phased array.

55. A system as in claim 50, wherein the vibrating surface comprises a transducer assembly which translates along an axial path in the container, wherein the axial path is parallel to the axis of the inner sleeve.

56. A system as in claim 44, wherein the container includes a rigid outer shell, wherein the inner sleeve is suspendable in the rigid outer shell which can be filled with an acoustically transmissive medium.

57. A system as in claim 44, wherein the vibrational energy is delivered by the vibrating surface under conditions comprising a frequency in the range from 30 kHz to 5 MHz and a mechanical index in the range from 0.5 to 4.

58. A system as in claim 57, wherein the conditions further comprise a duty cycle in the range from 0.1% to 50%.

59. A system as in claim 57, wherein the conditions further comprise a burst rate in the range from 0.1 kHz to 50 kHz and cycles per burst in the range from 2 to 10,000.
